(19) United States
(12) Patent Application Publication
(10) Pub. No.: US 2025/0372260 A1
(43) Pub. Date: Dec. 4, 2025

(54) INTELLIGENT DETECTION OF WELLNESS EVENTS USING MOBILE DEVICE SENSORS AND CLOUD-BASED LEARNING SYSTEMS

(71) Applicant: Alarm.com Incorporated, Tysons, VA (US)

(72) Inventors: Steve Chazin, Tysons, VA (US); Stephen Scott Trundle, Falls Church, VA (US); Daniel Todd Kerzner, McLean, VA (US)

(21) Appl. No.: 19/300,760

(22) Filed: Aug. 15, 2025

Related U.S. Application Data
(63) Continuation of application No. 17/167,746, filed on Feb. 4, 2021.
(60) Provisional application No. 62/970,149, filed on Feb. 4, 2020.

(51) Int. Cl. G16H 50/20; G06N 3/04; G06N 3/08; G08B 5/22

(52) U.S. Cl. CPC G16H 50/20; G06N 3/04; G06N 3/08; G08B 5/222

(57) ABSTRACT

Methods and systems, including computer programs encoded on a computer storage-medium, are disclosed for implementing intelligent detection of wellness events using mobile device sensors and cloud-based learning systems. A system obtains sensor data generated by sensors integrated in a mobile device of a user. A machine-learning (ML) engine of the system generates a predictive model that identifies behavioral trends of the user. The model is generated using a neural network trained to identify patterns representing user trends in the sensor data. Based on communications with the device, the model is used to generate activity profiles of the user from the behavioral trends. The model is used to detect abnormal events involving the user when a parameter value of the activity profile exceeds a threshold. Notifications directed to assisting the user with alleviating the abnormal event are generated after detecting the abnormal events.

### CROSS-REFERENCE TO RELATED APPLICATION

This application is a continuation of U.S. application Ser. No. 17/167,746, filed Feb. 4, 2021, now pending, which claims the benefit of U.S. Provisional Application No. 62/970,149, filed on Feb. 4, 2020, now expired, all of which are incorporated by reference.

### FIELD

This specification relates to sensors for a mobile device or property.

### BACKGROUND

Monitoring devices and sensors are often dispersed at various locations at a property, such as a home or commercial business. These devices and sensors can have distinct functions at different locations of the property. Some sensors at a property offer different types of monitoring and control functionality. The functionality afforded by these sensors and devices can be leveraged to monitor the wellness of an individual at a property or to control certain safety devices that may be located at the properties.

Events relating to the well-being of a person or pet that occurs in a home or property can affect the health and wellness of occupants at the home. In general, some of these events can be classified as an unintentional or uncontrolled movement towards the ground or lower level and are a public health concern that can cause hospitalization of individuals that are adversely affected. In some cases, events that involve more serious health-related incidents can have debilitating and sometimes fatal consequences for the individual. Earlier detection and reporting of events that occur at a property can improve health outcomes for the persons affected by the events.

Early efforts to detect incidents that adversely affect the well-being of a user have employed wearable technologies to capture user input (e.g., panic button press) or to characterize and classify movements and postures. While these technologies may demonstrate reasonable utility in ideal conditions, user non-compliance and health-related incapacitation reduce general efficacy of these approaches. Furthermore, an inability to verify incidence of an actual or suspected well-being event leads to inaccurate reporting and undesirable handling of potentially serious events.

### SUMMARY

This document describes techniques for ambient well-being (or wellness) monitoring using mobile/electronic devices and artificial intelligence (AI) functions enabled by a predictive model. More specifically, techniques are described for implementing a computing system that accurately detects wellness conditions of a person from a remote or standoff distance relative to a location of the person. In contrast to prior solutions that require a person to wear a dedicated personal safety device, the system described in this document avoids the need for a dedicated safety device by obtaining sensor data from existing suites of sensors that are integrated in mobile devices routinely used by the person. The ability of the system to monitor and determine an overall assessment of an individual's well-being is improved given additional information from a diversity of sensors. For example, the system may optionally obtain additional sensor data from an existing suite of sensors that are configured for, or installed in, a property monitoring system at the person's residence.

Based on analysis of these sensor streams, a predictive model can be generated to identify or detect activity patterns and behavioral trends of a person. Such patterns and trends can be used to determine an overall wellness condition of the person. Similarly, the patterns and trends can be indicative of a probable or impending wellness event of the person. Hence, the system can be configured to detect a well-being event, such as a fall or other important physical safety condition that can affect, or is currently affecting, the person. The system can also report that detected occurrence to the user or to a third party for assistance. For example, instead of providing reactive assistance, the system is configured to provide notifications and generate commands to proactively assist the person in preventing pending wellness issues.

In some examples, the system is configured to detect pending or current human health conditions based on predictive analysis of additional streams of sensor data obtained from sensors integrated in devices such as smartwatches and other wearables devices. The additional sensor streams can provide richer datasets for analysis by the system, which enables the system to better evaluate pending or current health conditions on behalf of a user or caregiver. For instance, the system can intervene in response to a heart arrhythmia event, detected low oxygen levels (COPD), detected low blood sugar (diabetes), or related adverse health/well-being events.

Other implementations of this and other aspects include corresponding systems, apparatus, and computer programs, configured to perform the actions of the methods, encoded on computer storage devices. A computing system of one or more computers or hardware circuits can be so configured by virtue of software, firmware, hardware, or a combination of them installed on the system that in operation cause the system to perform the actions. One or more computer programs can be so configured by virtue of having instructions that, when executed by data processing apparatus, cause the apparatus to perform the actions.

### BRIEF DESCRIPTION OF THE DRAWINGS

FIG. 1 shows a block diagram of an example property and computing system for intelligent detection of events relating to the well-being of a user.

FIG. 2 shows an example wellness dashboard and profile data associated with a user.

FIG. 3 shows an example graphical interface that includes activity data associated with a well-being of a user.

FIG. 4 shows an example process for performing intelligent detection of events relating to the well-being of a user.

FIG. 5 shows a diagram illustrating an example property monitoring system.

Like reference numbers and designations in the various drawings indicate like elements.

### DETAILED DESCRIPTION

A property, such as a house or a place of business, can be equipped with a property monitoring system having multiple sensors and electronic devices that interact to enhance the wellness and security of individuals at the property.

The property monitoring system may include sensors, such as motion sensors, camera/digital image sensors, temperature sensors, distributed about the property to monitor conditions at the property. In many cases, the monitoring system also includes a control unit and one or more controls which enable automation of various actions at the property. In general, a security, automation, or property monitoring system may include a multitude of sensors and devices that are placed at various locations of a property to perform specific functions. These sensors and devices interact with the control units to provide sensor data to a monitoring server and to receive commands from the monitoring server.

In addition to the multiple sensors and devices that may be included in the property monitoring system, a user's mobile device may also interact with the control units to provide sensor data to the monitoring server and to receive commands or alerts from the monitoring server. The commands and alerts can relate to detected events or assessments regarding an individual's well-being. In some cases, the event detections and assessments about an individual's well-being are determined using sensor data obtained from sensors of the user's mobile device. For example, the determinations may be computed independent of the sensor data generated by the multiple sensors and devices at a property.

In this context, systems and methods are described that provide improvements in monitoring a well-being of a user or conditions relating to the well-being of a user and for proactively responding to a potential or actual event involving the well-being of the user. The approaches described herein leverage sensors integrated in mobile devices such as smartphones, smartwatches, including other smart-wearable devices, to collect sensor data about a user. Because these mobile devices are often used across various age groups as a primary communication tool, the data generated by the sensors installed in these devices provide an effective method of determining the state (e.g., wellness state) of a person at a distance.

The property monitoring system described in this specification is configured to process sensor data obtained from a smartphone or smartwatch of a user to detect a significant event, such as a fall, and indicate to the user or a remote caregiver the need to take appropriate action. The system includes a cloud-based machine-learning engine that is operable to process the sensor data obtained from these mobile devices to detect unexpected or abnormal activity based on a user's normal behavioral patterns. The processes implemented at the machine-learning engine allow for the detection of a plethora of human activities that can signal a probable or impending health and wellness issue. The detected events may then be attended to by family members, a monitoring service, or an AI/virtual caregiver, before the event progresses to a medical emergency.

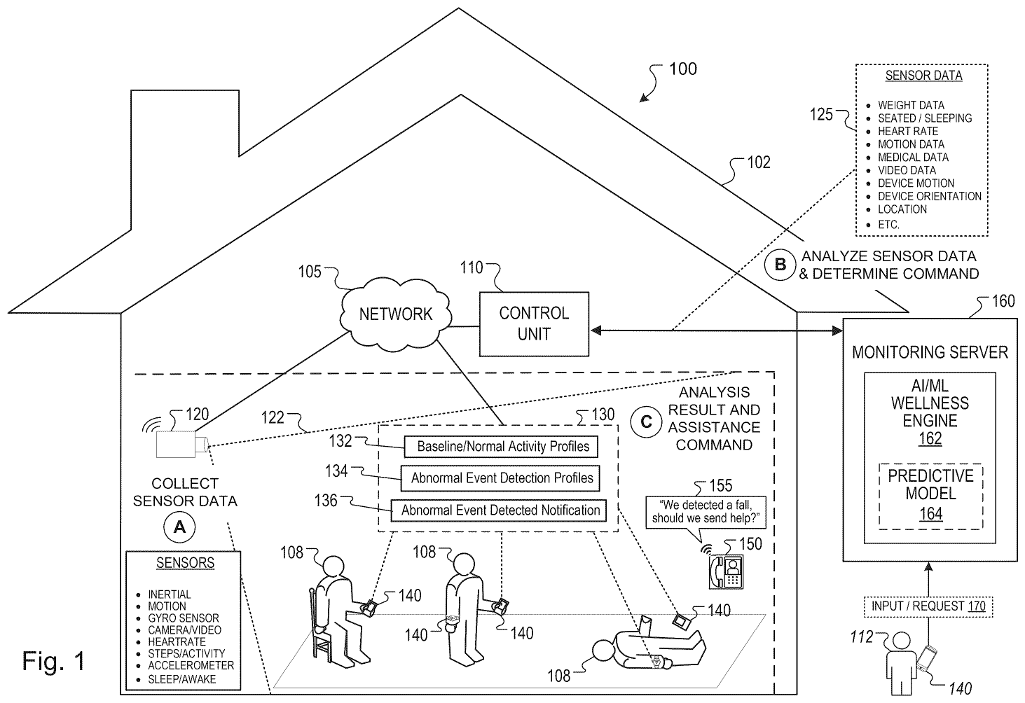

FIG. 1 shows a block diagram of an example monitoring system 100 (“system 100”) that can be used to perform one or more actions for securing a property 102 and for improving the safety and wellness of one or more occupants at the property 102. The property 102 may be, for example, a residence, such as a single family home, a townhouse, a condominium, or an apartment. In some examples, the property 102 may be a commercial property, a place of business, or a public property, such as a police station, fire department, or military installation.

The system 100 can include multiple sensors 120. One or more of the sensors 120 can be represented by various types of devices that are located at property 102. For example, a sensor 120 can be associated with a contact sensor that is operable to detect when a door or window is opened or closed. In some examples, a sensor 120 can be a bed/chair sensor that is operable to detect occupancy of a user 108 in a room or detect the user's sleep or rest cycle while at the property 102. Similarly, a sensor 120 can be associated with a video or image recording device located at the property 102, such as a digital camera or other electronic recording device configured to record video or images of the user 108 including other items in an example field of view 122.

One or more of the sensors 120 can be installed or otherwise integrated in various types of mobile devices 140 of a user 108 that is a resident or occupant of property 102. For example, at least one sensor 120 in the mobile device 140 can be an accelerometer or inertial sensor that is operable to detect rapid movement, vibration, or acceleration of the mobile device. In some examples, another sensor 120 in the mobile device 140 can be a gyroscopic sensor that is operable to measure an orientation of the mobile device or a rate of change in the orientation of the mobile device. A sensor 120 in the mobile device 140 can be associated with a transceiver of the mobile device 140 that receives and processes global positioning signals (GPS) to determine a location of the mobile device 140.

The mobile device 140 can be any one of the various types of known consumer electronic devices that may function as a primary communication tool for a user 108. In the example of the FIG. 1, the mobile device 140 can be represented as a smartphone or a smartwatch. In some implementations, the mobile device 140 can be any portable or handheld electronic device, such as a tablet device, an e-reader, a smart-wearable device, a smart speaker, an e-notebook, a gaming device (or console), or a laptop computer. In general, the mobile device 140 can include a variety of sensors that are typically integrated in these various types of consumer electronic devices.

The property monitoring system includes a control unit 110 that sends sensor data 125, obtained using sensors 120, to a remote monitoring server 160. In some implementations, the control units, monitoring servers, or other computing modules described herein are included as sub-systems of the monitoring system 100.

Control unit 110 can be located at the property 102 and may be a computer system or other electronic device configured to communicate with one or more of the sensors 120 to cause various functions to be performed for the property monitoring system or system 100. The control unit 110 may include a processor, a chipset, a memory system, or other computing hardware. In some cases, the control unit 110 may include application-specific hardware, such as a field-programmable gate array (FPGA), an application-specific integrated circuit (ASIC), or other embedded or dedicated hardware. The control unit 110 may also include software, which configures the unit to perform the functions described in this document.

The control unit 110 is configured to communicate with the mobile device 140 to obtain or pass sensor data 125 generated by sensors 120 in the mobile device 140 to the monitoring server 160 for analysis at the monitoring server 160. In this context, system 100 can be implemented, in part, by execution of program code in the form of an executable application, otherwise known as an “app,” that is installed and launched or executed from the mobile device 140. Upon execution, the app can then cause the mobile device 140 to establish a data connection with a computing server of system 100, e.g., a cloud-based server system, to transmit data signals to the computing server as well as to receive data signals from the computing server.

For example, a wellness monitoring app associated with the property monitoring system can be installed at mobile device 140. The wellness monitoring app causes the mobile device 140 to establish a data connection with the monitoring server 160 by way of the control unit 110 to transmit sensor data signals to the monitoring server 160 and to receive instructions and commands from the monitoring server 160. In some implementations, the wellness monitoring app causes the mobile device 140 to establish a data connection directly with the monitoring server 160 without using or relying on the control unit 110. In this manner, the mobile device 140 is operable to establish a direct connection with the monitoring server 160 to transmit sensor data signals to the monitoring server 160 and to receive instructions and commands from the monitoring server 160.

The wellness monitoring app may be granted permissions to access data associated with one or more sensor based applications that include functionality associated with accelerometers, gyroscopes, compasses, cameras, fitness activity, or other sensors 120 and applications installed or accessible at the mobile device 140. The monitoring server 160 is operable to receive sensor data 125 that is based on sensor data signals generated by one or more of the sensor devices 120 and corresponding sensor based applications on the mobile device 140. For example, sensor data 125 received by the monitoring server 160 can include device accelerometer data, device gyroscope data, location, health and fitness data, medical data, or any other sensor data signals associated with other movement or wellness based sensory applications of mobile device 140. In some implementations, the sensors of system 100 can optionally provide sensor data 125 that describes health information about an individual, such as age, weight, or height of the individual.

The sensors 120 communicate with the control unit 110, for example, through a network 105. The network 105 may be any communication infrastructure that supports the electronic exchange of sensor data 125 between the control unit 110 and the sensors 120. The network 105 may include a local area network (LAN), a wide area network (WAN), the Internet, or other network topology. In some implementations, the sensors 120 can receive, via network 105, a wireless (or wired) signal that controls operation of each sensor 120. For example, the signal can cause the sensors 120 to initialize or activate to sense activity at the property 102 and generate sensor data 125. The sensors 120 can receive the signal from monitoring server 160 or from control unit 110 that communicates with monitoring server 160, or from a predictive model 164 accessible by the monitoring server 160. In the example of FIG. 1 the predictive model 164 is shown as being accessible via the monitoring server 160, but as described below, the predictive model 164 can be implemented entirely at the mobile device 140 independent of network 105 or the monitoring server 160.

The monitoring server 160 is configured to pull, obtain, or otherwise receive different types of sensor data 125 from one or more of the various types of sensors 120, for example, using the control unit 110. The monitoring server 160 includes, or is configured to access, a machine-learning engine 162 (described below) that is operable to process and analyze the obtained sensor data 125. In response to analyzing the new data using the wellness engine 162, the monitoring server 160 can detect or determine that an abnormal condition may be affecting or is likely to affect an individual at the property 102.

As noted above, the machine-learning engine 162 is operable to process sensor data 125 obtained from the sensors 120 to determine conditions associated with an overall wellness or fitness of a person or individual at the property 102. In some implementations, the sensor data 125 is obtained using certain types of sensors 120 that are integrated in the mobile device 140, sensors 120 that are installed in different sections of the property 102, or both. The monitoring server 160 and machine-learning engine 162 correlates and analyzes the generated sensor data 125 with other wellness information received for the user 108 to determine activities and behavioral trends of the user 108 that indicate conditions associated with the overall wellness of the individual.

The machine-learning engine 162 is configured to process the sensor data 125 using a neural network of the machine-learning engine. The neural network may be an example artificial neural network, such as a deep neural network (DNN) or a convolutional neural network (CNN). In general, neural networks are machine learning models that employ one or more layers of operations to generate an output, e.g., a predicted inference or classification, for a received input. Some neural networks include one or more hidden layers in addition to an output layer. The output of each hidden layer is used as input to the next layer in the network, e.g., the next hidden layer or the output layer of the network. Some or all of the layers of the network generate an output from a received input in accordance with current values of a respective set of parameters.

A neural network having multiple layers can be used to compute inferences. For example, given an input, the neural network can compute an inference for the input. The neural network computes this inference by processing the input through each of the layers of the neural network. In general, prior to computing inferences the neural network may be first trained on a sample or training dataset by processing the dataset through each of the layers of the neural network. In some implementations, the neural network is implemented on a hardware circuit, such as a special-purpose processor of the monitoring server 160. For example, the monitoring server 160 may be configured to include or access a hardware machine-learning accelerator that is a processor microchip operable to run various types of machine-learning models.

The sensor data 125 obtained from each of the sensors 120 that are integrated in the mobile device 140, and/or each of the sensors 120 installed at the property 102, can be processed by a neural network to train the neural network based on an example training algorithm. The machine-learning engine 162 processes the sensor data 125 to train the neural network by identifying patterns representing user trends in the sensor data 125. During training of the neural network, the identification of the patterns and relationships between variables (or latent variables) in the sensor data 125 may be based on one or more training algorithms.

In some cases, training the neural network to compute inferences or predictions represents a process of generating a predictive model. For example, the machine-learning engine 162 can generate a predictive model 164 in response to processing a representative sampling of sensor data 125 to train the neural network. In some implementations, the system 100 includes a training phase that is run for a particular duration or time period to collect and process sensor data 125 that is used to generate predictive model 164. For example, the machine-learning engine 162 is operable to run or execute the training phase to generate representative samples of sensor data 125 for generating the predictive model 164.

The training phase may be run continuously or intermittently for a predetermined duration, such as 5 days, 10 days, or 30 days. In some cases, parameters that are associated with the training phase, such as duration, frequency, or types of sensor data and feature types, may be set by the user 108 or an end-user 112. For example, the parameters for the training phase may be set using an optional security panel 150 at the property 102 or using the wellness monitoring app.

The predictive model 164 is configured to identify various behavioral trends of the user. For example, the training phase allows the machine-learning engine 162 to observe and learn various tendencies and characteristics of the user 108 based on analysis of data values in the representative samples of sensor data 125 that are processed during the training phase. The sample datasets processed during training can include information about a fitness, wellness, or medical status of a person. For example, the predictive model 164 can be tuned to detect, identify, or determine certain patterns, trends, and tendencies of a user 108 (collectively “behavioral trends”).

After being initially trained, the predictive model 164 is configured to identify multiple behavioral trends of the user 108. For example, the predictive model 164 is configured to identify one or more behavioral trends that indicate details about the respiration, heart rate, or blood pressure of the user 108. In some examples, the predictive model 164 is configured to identify one or more behavioral trends that provide details about how user 108 moves about the property 102 or the types of activities that are typically performed by the user 108 while at the property 102.

For example, the behavioral trends may reveal how often the user frequents a particular room (e.g., the bedroom or bathroom) at the property 102, how often a user charges or unlocks their phone, the general locations of the user's mobile device/phone, or the number of steps and general activity level of the user as tracked by sensors of the user's mobile device. Hence, various behavioral trends can be identified or detected based on analysis of sensor data 125 by the predictive model 164, the machine-learning engine 162, the monitoring server 160, or combinations of each.

The system 100 uses the predictive model 164 to generate a wellness profile 130 for the user 108 based on the various types of behavioral trends that are identified about the user 108. The wellness profile 130 can include one or more activity profiles 132, one or more event detection profiles 134, and one or more detected events 136.

The activity profiles 132 include parameters and data values that are indicative of baseline or normal activity of the user 108. The activity profiles can indicate daily or weekly actions or tendencies of the user 108 relative to the user's mobile device 140 or items at the property 102. For example, the parameters and data values of a first activity profile 132 can indicate that the user routinely handles their mobile device 140 every 20 to 30 minutes and consistently keeps their the charge level of the battery voltage in the mobile device 140 above 50%.

The event detection profiles 134 include threshold data values for certain parameters that can be used to trigger detection of an event relating to the safety, health, or wellness of the user 108. The event detection profiles 134 can be abnormal event detection profiles that have threshold values for triggering detection of certain abnormal events involving the user, such as events that may be detrimental to the health and wellness of the user 108. The event detection profiles 134 can be used to detect certain deviations from the baseline or normal activity of the user 108 that warrant the triggering or detection of a wellness event.

For example, the parameters and data values of a first event detection profile 134 can be set to trigger an event detection when the user hasn't handled their device for 2 hours based on activity profile data that indicates the user 108 should be routinely handling mobile device 140 every 20 to 30 minutes. The detected events 136 include information about current or past events (e.g., abnormal events) detected for a user 108 or event notifications generated for a user 108. In some implementations, the detected events 136 can include a listing of events that have been detected for the user 108.

FIG. 1 includes stages A through C, which represent a flow of data.

In stage (A), each of the one or more sensors 120 generate sensor data 125 including parameter values that describe different types of sensed activity at the property 102, such as activity involving the user's interaction within and handling of mobile device 140. In some implementations, the control unit 110 (e.g., located at the property 102) collects and sends the sensor data 125 to the remote monitoring server 160 for processing and analysis at the monitoring server. The sensor data 125 can include parameter values that indicate a weight of a person, a pet's location relative to a geo-fence at the property 102, how a user 108 enters or exists a particular room at the property 102, the user's heartrate as indicated by a smartwatch or mobile device 140. The sensor data 125 can also include parameter values that indicate sensed motion or force distribution when the person is sitting in a chair or standing up from being seated in a chair, medical conditions of the person, a body temperature of the person, or images/videos of the person.

In stage (B), the monitoring server 160 receives or obtains sensor data 125 from the control unit 110. As discussed above, the monitoring server 160 can communicate electronically with the control unit 110 through a wireless network, such as a cellular telephony or data network, through any of various communication protocols (e.g., GSM, LTE, CDMA, 3G, 4G, 5G, 802.11 family, etc.). In some implementations, the monitoring server 160 receives or obtains sensor data 125 from the individual sensors rather than from control unit 110. In some implementations, the monitoring server 160 receives or obtains sensor data 125 directly from the individual sensors integrated in a user's mobile device rather than from the control unit 110 or from other sensors present at the property 102.

In stage (C), the monitoring server 160 analyzes the sensor signal data 125 and/or other property data received from the control unit 110 or directly from sensors/devices 120 located at the property 102. As indicated above, the monitoring server 160 analyzes the sensor data 125 to determine wellness attributes of a person, including one or more conditions associated with overall fitness or wellness of a person, and to determine whether an event notification should be triggered to inform at least an end-user 112 about an abnormal event involving the user.

The predictive model 164 is operable to analyze parameter values that reveal routine activities that are typically performed by the user 108. Analysis of the parameters can reveal deviations from those routine actions that indicate a potential abnormal event, such as a sudden fall at the property 102 or a prolonged period of inactivity that may be indicative of a serious medical emergency. In some implementations, the monitoring server 160 uses encoded instructions of the predictive model 164 to measure, infer, or otherwise predict potential abnormal health events that may negatively affect the user 108. As noted above, in some implementations, the predictive model 164 is implemented entirely on the user's mobile device 140 and the monitoring server 160 may interact with the predictive model 164 at the mobile device 140 to predict the potential abnormal health events. Each of the predictions about current or potential abnormal events are uniquely specific to that user, rather than to a larger population.

In some cases, the techniques described herein for detecting abnormal health events that are affecting, or could affect, a user do not require additional sensors beyond those that are already part of a smartphone such as mobile device 140. Rather, additional sensors 120, such as those installed at the property 102, provide supplemental data inputs that are processed by the machine-learning engine 162 and the predictive model 164 to improve upon the accuracy of the predictive outputs generated by these ML systems. As such, the disclosed techniques do require a “property monitoring system” to operate, but can benefit from one.

The machine-learning engine 162 is operable to reference templates of normal activity for individuals with similar characteristics to the user 108. For example, if the user is a male, age 65, and living in San Francisco, CA, then the machine-learning engine 162 is operable to reference one or more templates for males (e.g., age 62-67) in and around the San Francisco area to determine parameters and data values that can be used to determine one or more sets of profile data 130 for the user 108. For example, the machine-learning engine 162 may reference the templates to determine reasonable ranges for threshold values based on other indications of routine/normal activity of other similar users 108. In some implementations, the referencing of templates that are accessible by the machine-learning engine 162 is based on a bias function encoded at the monitoring server 160.

The predictive model 164 is operable to generate a notification directed to assisting the user 108 with alleviating the abnormal event. For example, in response to detecting that user 108 suddenly fell (e.g., an abnormal event) at the property 102, the system 100 can initiate a voice connection between the property 102 and a central monitoring station that monitors the property. For example, a two-way voice connection can be used to transmit a voice communication 155 from an end-user 112 to the user 108 indicating that a fall was detected. In some implementations, the predictive model 164 is operable to generate a notification to first responders to inform the first responders that a fall was detected at the property 102. The notification to the first responders may cause the first responders to arrive at the property 102 to assist the user 108 with obtaining medical treatment in response to the fall. The predictive model 164 is also operable to generate a notification to a user's loved ones or family members allowing the family members to stay abreast of changes to the well-being of user 108 before those changes become a more serious issue or health concern.

The voice communication can be output at the property 102 via a speaker integrated in the security panel 150. The voice communication can be also output at the property 102 via the mobile device 140 of the user 108. In some implementations, the two-way voice connection between the central monitoring station and the property 102 is initiated or established using a cellular modem integrated at an optional security panel 150 at the property 102. The two-way voice connection can be used to notify the user 108 that an end-user 112 has detected a fall at the property 102. The notification can inform the user 108 that help, e.g., first responders, is on the way. Alternatively, the two-way voice connection can be used to pass a reply from user 108 as voice data to the end-user 112.

Though the stages are described above in order of (A) through (C), it is to be understood that other sequencings are possible and disclosed by the present description. For example, in some implementations, the monitoring server 160 may receive sensor data 125 from the control unit 110. The sensor data 125 can include both sensor status information and usage data/parameter values that indicate or describe specific types of sensed activity for each sensor 120. In some cases, aspects of one or more stages may be omitted. For example, in some implementations, the monitoring server 160 may receive and/or analyze sensor data 125 that includes only usage information rather than both sensor status information and usage data.

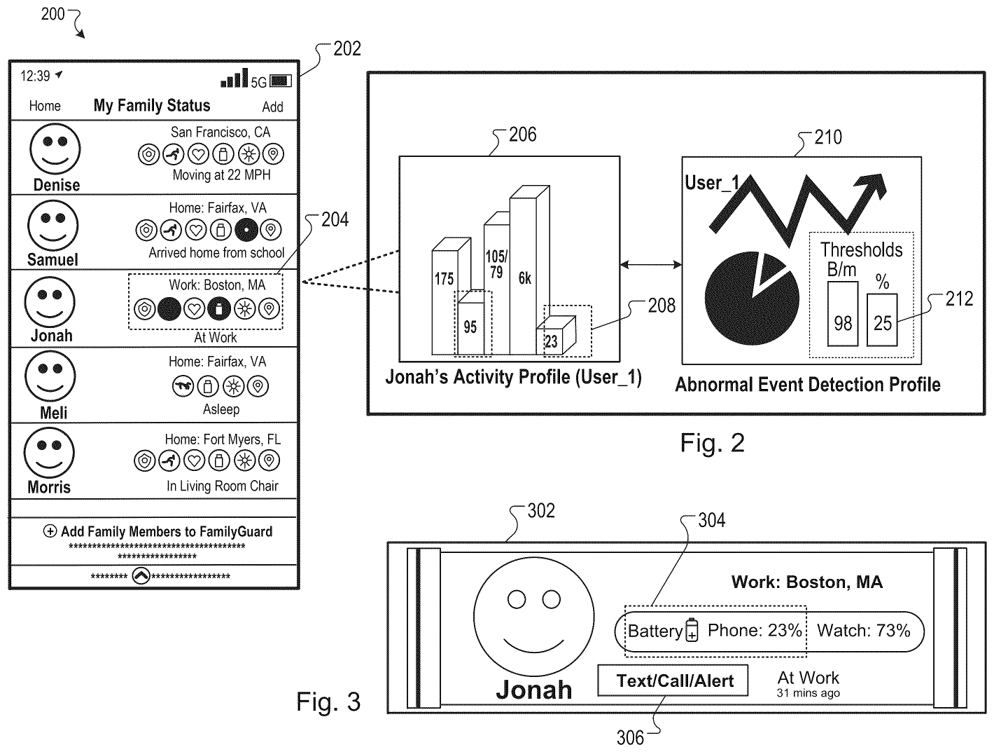

FIG. 2 shows an example wellness dashboard 200 and at least one graphical interface 202 that includes display icons 204 that indicate profile data associated with a user 108 (e.g., Jonah). The dashboard 200 can be one of multiple graphical interfaces that are generated by the wellness monitoring app described above with reference to FIG. 1. In some implementations, the wellness monitoring app may be sub-program or sub-system of the monitoring system 100. The display icons 204 of the dashboard 200 provide color coded indications of a wellness status or condition of the user 108. For example, the display icons 204 are operable to provide an indication of abnormal activity of the user 108 based on a particular color of an icon.

The system 100 can use the predictive model 164 to generate a graphical interface configured to present information indicating a current health condition of the user based on inferences computed by the predictive model. The predictive model 164 is operable to dynamically adjust the current health condition of the user to reflect determinations that the user has engaged in activity or inactivity indicative of the event that is detrimental to a health condition of the user. The graphical interface (e.g., dashboard 200) can be used to display the activity profile of the user. For example, the graphical interface is operable to overlay one or more icons on the activity profile to indicate: i) the current health condition of the user, ii) detection of the abnormal event, and iii) the determination that the user has engaged in activity or inactivity indicative of the event that is detrimental to the health condition of the user.

For example, a red heart icon may indicate that the user 108 has an elevated heart rate that is related to a medical emergency. In some examples, the dashboard 200 is configured to include glanceable color coded icons that indicate all is normal/well with the well-being status of user 108. In some implementations, an example AI construct generated based on the predictive model 164 can be engaged to watch over or monitor user 108 on behalf of a caregiver that is responsible for the care and well-being of user 108.

The wellness monitoring app is installed at mobile device 140 of the user 108, such as tablet or smartphone owned by the user 108. In some cases, a first version of the wellness monitoring app may be installed on the user's smartphone while a second, different version of the wellness monitoring app may be installed in the user's smartwatch. In some implementations, each of the first and second versions of the wellness monitoring app is operable to communicate with an optional security panel 150 at the property 102, to adjust settings of the security panel 150, or to exchange voice or data signals.

The system 100 uses the activity profile 206 or discrete parameters of the activity profile 206 to generate the wellness dashboard 200 for the user. The wellness dashboard 200 may be presented to an end-user 112 as a graphical user interface (GUI) 202 of the wellness monitoring app. As noted above, the predictive model 164 can include computing logic for generating one or more abnormal event detection profiles 210. Each profile 210 can have various threshold values 212 for triggering detection of certain abnormal events involving the user 108, such as events that may be detrimental to the health and wellness of the user 108. In the example of FIG. 2, the predictive model 164 can generate an abnormal event detection profiles 210 that includes an example threshold value for the user's heart rate measured in beats/minute (b/m) and a device charging threshold that specifies a minimum charge level of the device.

The event detection profiles 134 can include various threshold data values for certain parameters that can be used to trigger detection of an event relating to the safety, health, or wellness of the user 108. For example, at least parameter can be a location parameter that triggers an alert when the user travels a threshold distance from the property, e.g., 75 feet. The event detection profiles 134 can be abnormal event detection profiles that have threshold values for triggering detection of certain abnormal events involving the user, such as events that may be detrimental to the health and wellness of the user 108. The event detection profiles 134 can be used to detect certain deviations from the baseline or normal activity of the user 108 that warrant the triggering or detection of a wellness event.

For example, the parameters and data values of a first event detection profile 134 can be set to trigger an event detection when the user hasn't handled their device for 2 hours based on activity profile data that indicates the user 108 should be routinely handling mobile device 140 every 20 to 30 minutes. Similarly, the parameters and data values of a second event detection profile 134 can be set to trigger an event detection when the charge level of the battery voltage in mobile device 140 is below 25% based on activity profile data that indicates the user 108 consistently keeps the charge level above 50%. In some implementations, the predictive model 164 uses machine-learning logic to process multiple different variables (e.g., heart rate, steps, calories expended, user location, device charge level, blood pressure, etc.) and to determine an optimal weighting on the variables to generate an activity profile and corresponding detection thresholds that most accurately represent activity levels of the user.

The system 100 can generate multiple different signals corresponding to a second data type at least by converting a first data signal of a first data type to a second data signal of a second data type. For example, the system 100 can convert one type of data (e.g., battery charge percentage) into another type of data (e.g. wellness signal). In this context, a person who charges their phone regularly every night and takes the phone off the charger each morning may provide a proxy for “time asleep.” Based on this proxy, the system 100 may generate a corresponding sleep duration data signal that represents the user's “time asleep.”

In some implementations, the system 100 combines the proxy signal with one or more other signals, such as heart rate below a threshold heart rate, to obtain a more reliable indication of the user's time asleep. Relatedly, when a person who religiously charges their phone whenever it drops below X % suddenly stops doing so, the system 100 can use this indicator to generate a corresponding signal for reporting a sudden wellness change. This particular signal may be grouped with one or more other signals (e.g., location, motion, recent steps, heart rate etc.) to obtain a more reliable indication of a sudden wellness change.

FIG. 3 shows an example graphical interface 302 that includes a display icon 304 that corresponds to a battery charge level 208 (23%) in the activity profile 206. The icon 304 may be color coded in the interface 302 to indicate detection of an abnormal wellness event involving a user 108 based on the parameter value for the battery charge level 208, e.g., 23%, in the activity data for the user. For example, the charge level of 23% indicated by the icon 304 is below the 25% threshold 212, which can trigger detection of an abnormal wellness event involving a user 108.

In general, an end-user 112 can use one or more of the graphical interfaces of the wellness dashboard 200, e.g., graphical interface 302, to view information indicating a wellness status of the user 108. The system 100 is configured to process data associated with the activity profile 206 of the user 108 to determine a wellness condition of the user or a prospective wellness condition of the user 108. The wellness condition may indicate whether the user has been or is engaging in behaviors and actions that are consistent with normal activity of the user 108.

For instance, the mere act of a user 108 (e.g., an elderly person) is charging their phone/mobile device 140 every day suggests that the user 108 is healthy enough to perform the act of attending to their mobile device 140 or smartphone. Such wellness signals may indicate that the elderly user 108 is has normal or generally healthy wellness status. Similarly, when a young person who typically unlocks their smartphone 140 or mobile device multiple times every hour, e.g., during a particular time period of the day, is noticed to have not unlocked their mobile device 140 for the past six hours, this may prompt the system 100 to generate a check-in notification to the user 108 to determine if the user 108 has experienced an adverse event.

In some implementations, the system 100 is configured to continuously or iteratively assess a users' normal or expected daily phone motion. In addition to, or concurrent with, this assessment, the system 100 can also check whether the mobile device 140 is at a location outside of an expected area at certain times of the day. Based on these checks and assessments, the system 100 can then determine when activity levels associated with the user are atypical and indicative of an adverse health event that is affecting the user. The system 100 generates a wellness alert/notification 306 for an end-user 112 in response to receiving user input from the end-user in the form of a request or command 170. For example, the end-user 112 may submit a request or command 170 to system 100 that causes the predictive model 164 to obtain wellness data about the user.

Assuming the user has consented to location monitoring and configured any related privacy settings, using the machine-learning engine 162 and the predictive model(s) 164, the system 100 is operable to learn a user's typical or expected areas and locations of travel. Based on these learned areas and/or locations, the system 100 can detect deviations from the expected behavior, such as when the user has deviated from their expected routes of travel, and report on the detected deviation.

In some implementations, the system 100 generates an automatic geofence based on locations and routes of travel that machine-learning logic of the system has learned are specific or routine for the user or caregiver. Based on this learned behavior/model output, the system 100 is operable to alert a user or caregiver in response to determining that the user has traveled to an unexpected location. This intelligence logic of the system 100 can also extend to other location-aware devices that may be attached to, or worn by, the user, such as a mobile Personal Emergency Response (mPERS) device, GPS trackers, or a smartwatch. The monitoring server 160 may be configured to interact with each of these devices to receive sensor data 125 or location information that can be processed by the machine-learning engine 162 to generate the geofence and perform location related computations to detect user deviations from expected routes of travel.

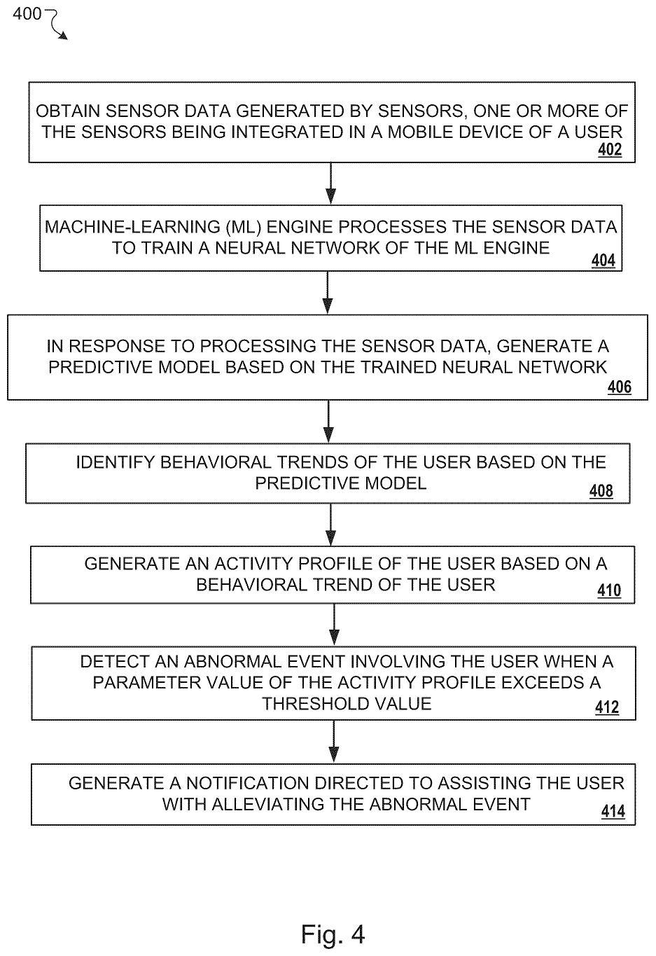

FIG. 4 shows an example process 400 for performing intelligent detection of wellness events relating to a user. Process 400 can be implemented or performed using the systems described in this document. For example, the process 400 may be embodied in a set of executable program instructions stored on a computer-readable medium, such as one or more disk drives, of a computing system of the monitoring server 160. In general, descriptions of process 400 may reference one or more of the above-mentioned computing resources of system 100. In some implementations, one or more steps of process 400 are enabled by programmed instructions executed by processing devices of the sensors, mobile devices, and systems described in this document.

Referring now to process 400, system 100 obtains sensor data generated by multiple sensors that interact or communicate within the system (402). In some cases, a first portion of the multiple sensors that generate sensor data and communicate within the system 100 are integrated in one or more mobile devices of a user, whereas a second portion of the multiple sensors that generate sensor data and communicate within the system 100 are installed, integrated, or otherwise located at the property 102.

For example, the system 100 can obtain, from sensors 120 integrated in a mobile device 140 of user 108, sensor data 125 that indicates a location of the user at the property 102 based on a location of the mobile device 140 or a charge level of the battery voltage in the mobile device 140. The system 100 can also obtain, from sensors 120 installed or located at the property 102, sensor data 125 such as video data or motion data indicating the user 108 is moving about the property 102.

A machine-learning engine of the system 100 processes the sensor data using a neural network of the machine-learning engine (404). More specifically, the machine-learning engine 162 processes the sensor data 125 to train the neural network by identifying patterns representing user trends in the sensor data. For example, the sensor data 125 obtained from each of the sensors 120 that are integrated in the mobile device 140, and/or each of the sensors 120 installed at the property 102, can be processed by a neural network to train the neural network based on an example training algorithm.

In response to processing the sensor data, the system 100 generates a predictive model based on the trained neural network (406). For example, the machine-learning engine 162 of system 100 generates a predictive model 164 that is based on the trained neural network. In some implementations, the predictive model 164 is generated based on a training phase that is run at the machine-learning engine 162 for a predetermined duration. The predictive model 164 is configured to identify a plurality of behavioral trends of the user.

In this manner, the system 100 is configured to identify, using at least the predictive model, one or more behavioral trends of the user (408). For example, the system 100 uses the machine-learning engine 162 and the predictive model 164 to identify multiple behavioral trends of the user corresponding to different types of actions and tendencies of the user.

The system 100 generates an activity profile of the user (410). System 100 generates an activity profile of the user with reference to inferences and predictions computed about the user by the predictive model 164 generated from the trained neural network. For example, the predictive model 164 is operable to generate an activity profile of the user based on one or more behavioral trends of the user 108. At least one of the behavioral trends used to generate the activity profile is indicative of normal activity of the user 108. In some examples, the normal activity of the user 108 can correspond to routine or expected actions that are typically performed by the user 108.

The system 100 detects an abnormal event involving the user (412). More specifically, the system 100 uses the predictive model 164 to detect an abnormal event involving the user 108 based on one or more parameters of the activity profile. For example, the system 100 uses the predictive model 164 to analyze parameters and corresponding parameter values of the activity profile. The predictive model 164 is operable to detect an abnormal event involving the user 108 when a parameter value of the activity profile of the user exceeds a threshold parameter value.

The system 100 generates a notification directed to assisting the user with alleviating the abnormal event (414). In response to detecting the abnormal event, the system 100 can use the predictive model 164 to generate a notification directed to assisting the user with alleviating the abnormal event. For example, in response to detecting an abnormal event corresponding to user 108 suddenly falling at the property 102, the system 100 initiates a two-way voice connection between a central monitoring station and the property 102. The two-way voice connection can be used to provide voice notifications from end-user 112 to the user 108 indicating that a fall was detected.

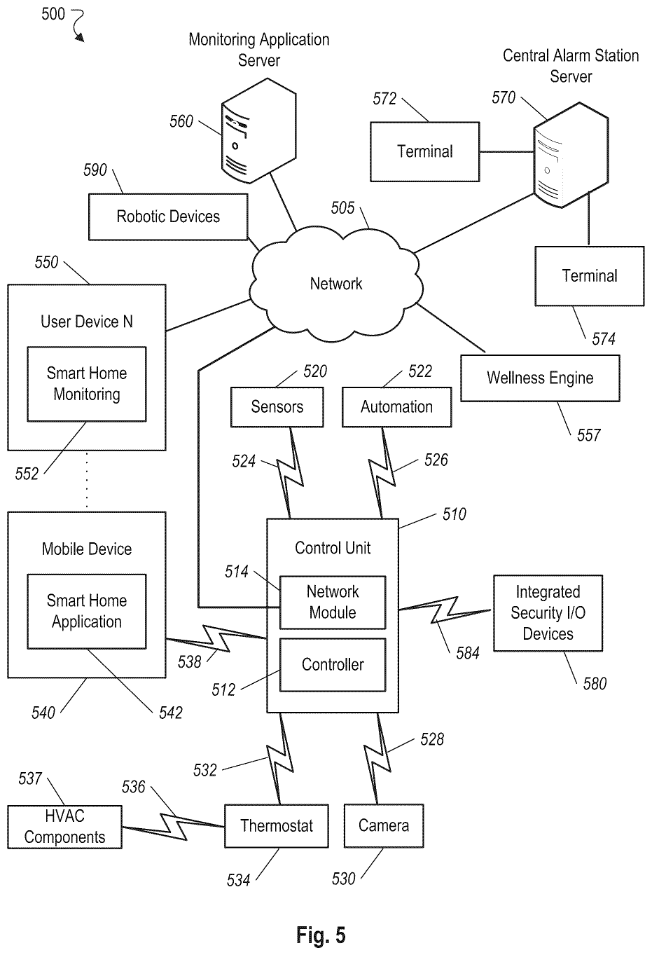

FIG. 5 is a diagram illustrating an example of a property monitoring system 500. The electronic system 500 includes a network 505, a control unit 510 (optional), one or more user devices 540 and 550, a monitoring server 560, and a central alarm station server 570. In some examples, the network 505 facilitates communications between the control unit 510, the one or more user devices 540 and 550, the monitoring server 560, and the central alarm station server 570.

The network 505 is configured to enable exchange of electronic communications between devices connected to the network 505. For example, the network 505 may be configured to enable exchange of electronic communications between the control unit 510, the one or more user devices 540 and 550, the monitoring server 560, and the central alarm station server 570. The network 505 may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. Network 505 may include multiple networks or subnetworks, each of which may include, for example, a wired or wireless data pathway. The network 505 may include a circuit-switched network, a packet-switched data network, or any other network able to carry electronic communications (e.g., data or voice communications). For example, the network 505 may include networks based on the Internet protocol (IP), asynchronous transfer mode (ATM), the PSTN, packet-switched networks based on IP, x.25, or Frame Relay, or other comparable technologies and may support voice using, for example, VoIP, or other comparable protocols used for voice communications. The network 505 may include one or more networks that include wireless data channels and wireless voice channels. The network 505 may be a wireless network, a broadband network, or a combination of networks including a wireless network and a broadband network.

The control unit 510 includes a controller 512 and a network module 514. The controller 512 is configured to control a control unit monitoring system (e.g., a control unit system) that includes the control unit 510. In some examples, the controller 512 may include a processor or other control circuitry configured to execute instructions of a program that controls operation of a control unit system. In these examples, the controller 512 may be configured to receive input from sensors, flow meters, or other devices included in the control unit system and control operations of devices included in the household (e.g., speakers, lights, doors, etc.). For example, the controller 512 may be configured to control operation of the network module 514 included in the control unit 510.

The network module 514 is a communication device configured to exchange communications over the network 505. The network module 514 may be a wireless communication module configured to exchange wireless communications over the network 505. For example, the network module 514 may be a wireless communication device configured to exchange communications over a wireless data channel and a wireless voice channel. In this example, the network module 514 may transmit alarm data over a wireless data channel and establish a two-way voice communication session over a wireless voice channel. The wireless communication device may include one or more of a LTE module, a GSM module, a radio modem, cellular transmission module, or any type of module configured to exchange communications in one of the following formats: LTE, GSM or GPRS, CDMA, EDGE or EGPRS, EV-DO or EVDO, UMTS, or IP.

The network module 514 also may be a wired communication module configured to exchange communications over the network 505 using a wired connection. For instance, the network module 514 may be a modem, a network interface card, or another type of network interface device. The network module 514 may be an Ethernet network card configured to enable the control unit 510 to communicate over a local area network and/or the Internet. The network module 514 also may be a voice band modem configured to enable the alarm panel to communicate over the telephone lines of Plain Old Telephone Systems (POTS).

The control unit system that includes the control unit 510 includes one or more sensors. For example, the monitoring system may include multiple sensors 520. The sensors 520 may include a lock sensor, a contact sensor, a motion sensor, or any other type of sensor included in a control unit system. The sensors 520 also may include an environmental sensor, such as a temperature sensor, a water sensor, a rain sensor, a wind sensor, a light sensor, a smoke detector, a carbon monoxide detector, an air quality sensor, etc. The sensors 520 further may include a health monitoring sensor, such as a prescription bottle sensor that monitors taking of prescriptions, a blood pressure sensor, a blood sugar sensor, a bed mat configured to sense presence of liquid (e.g., bodily fluids) on the bed mat, etc. In some examples, the health monitoring sensor can be a wearable sensor that attaches to a user in the home. The health monitoring sensor can collect various health data, including pulse, heart-rate, respiration rate, sugar or glucose level, bodily temperature, weight or body mass levels of a user, pulse oximetry, or motion data. The sensors 520 can also include a radio-frequency identification (RFID) sensor that identifies a particular article that includes a pre-assigned RFID tag as well as Cat-M cellular and Bluetooth Low Energy (BLE) related sensors.

The control unit 510 communicates with the home automation controls 522 and a camera 530 to perform monitoring. The home automation controls 522 are connected to one or more devices that enable automation of actions in the home. For instance, the home automation controls 522 may be connected to one or more lighting systems and may be configured to control operation of the one or more lighting systems. Also, the home automation controls 522 may be connected to one or more electronic locks at the home and may be configured to control operation of the one or more electronic locks (e.g., control Z-Wave locks using wireless communications in the Z-Wave protocol). Further, the home automation controls 522 may be connected to one or more appliances at the home and may be configured to control operation of the one or more appliances. The home automation controls 522 may include multiple modules that are each specific to the type of device being controlled in an automated manner. The home automation controls 522 may control the one or more devices based on commands received from the control unit 510. For instance, the home automation controls 522 may cause a lighting system to illuminate an area to provide a better image of the area when captured by a camera 530.

The camera 530 may be a video/photographic camera or other type of optical sensing device configured to capture images. For instance, the camera 530 may be configured to capture images of an area within a building or home monitored by the control unit 510. The camera 530 may be configured to capture single, static images of the area and also video images of the area in which multiple images of the area are captured at a relatively high frequency (e.g., thirty images per second). The camera 530 may be controlled based on commands received from the control unit 510.

The camera 530 may be triggered by several different types of techniques, including WiFi motion or Radar based techniques. For instance, a Passive Infra-Red (PIR) motion sensor may be built into the camera 530 and used to trigger the camera 530 to capture one or more images when motion is detected. The camera 530 also may include a microwave motion sensor built into the camera and used to trigger the camera 530 to capture one or more images when motion is detected. The camera 530 may have a “normally open” or “normally closed” digital input that can trigger capture of one or more images when external sensors (e.g., the sensors 520, PIR, door/window, etc.) detect motion or other events. In some implementations, the camera 530 receives a command to capture an image when external devices detect motion or another potential alarm event. The camera 530 may receive the command from the controller 512 or directly from one of the sensors 520.

In some examples, the camera 530 triggers integrated or external illuminators (e.g., Infra-Red, Z-wave controlled “white” lights, lights controlled by the home automation controls 522, etc.) to improve image quality when the scene is dark. An integrated or separate light sensor may be used to determine if illumination is desired and may result in increased image quality.

The camera 530 may be programmed with any combination of time/day schedules, system “arming state”, or other variables to determine whether images should be captured or not when triggers occur. The camera 530 may enter a low-power mode when not capturing images. In this case, the camera 530 may wake periodically to check for inbound messages from the controller 512. The camera 530 may be powered by internal, replaceable batteries if located remotely from the control unit 510. The camera 530 may employ a small solar cell to recharge the battery when light is available. Alternatively, the camera 530 may be powered by the controller's 512 power supply if the camera 530 is co-located with the controller 512.

In some implementations, the camera 530 communicates directly with the monitoring server 560 over the Internet. In these implementations, image data captured by the camera 530 does not pass through the control unit 510 and the camera 530 receives commands related to operation from the monitoring server 560.

The system 500 also includes thermostat 534 to perform dynamic environmental control at the home. The thermostat 534 is configured to monitor temperature and/or energy consumption of an HVAC system associated with the thermostat 534, and is further configured to provide control of environmental (e.g., temperature) settings. In some implementations, the thermostat 534 can additionally or alternatively receive data relating to activity at a home and/or environmental data at a home, e.g., at various locations indoors and outdoors at the home. The thermostat 534 can directly measure energy consumption of the HVAC system associated with the thermostat, or can estimate energy consumption of the HVAC system associated with the thermostat 534, for example, based on detected usage of one or more components of the HVAC system associated with the thermostat 534. The thermostat 534 can communicate temperature and/or energy monitoring information to or from the control unit 510 and can control the environmental (e.g., temperature) settings based on commands received from the control unit 510.

In some implementations, the thermostat 534 is a dynamically programmable thermostat and can be integrated with the control unit 510. For example, the dynamically programmable thermostat 534 can include the control unit 510, e.g., as an internal component to the dynamically programmable thermostat 534. In addition, the control unit 510 can be a gateway device that communicates with the dynamically programmable thermostat 534. In some implementations, the thermostat 534 is controlled via one or more home automation controls 522.

A module 537 is connected to one or more components of an HVAC system associated with a home, and is configured to control operation of the one or more components of the HVAC system. In some implementations, the module 537 is also configured to monitor energy consumption of the HVAC system components, for example, by directly measuring the energy consumption of the HVAC system components or by estimating the energy usage of the one or more HVAC system components based on detecting usage of components of the HVAC system. The module 537 can communicate energy monitoring information 556 and the state of the HVAC system components to the thermostat 534 and can control the one or more components of the HVAC system based on commands received from the thermostat 534.

The system 500 includes one or more predictive wellness engines 557. Each of the one or more predictive wellness engine 557 connects to control unit 510, e.g., through network 505. The predictive wellness engines 557 can be computing devices (e.g., a computer, microcontroller, FPGA, ASIC, or other device capable of electronic computation) capable of receiving data related to the sensors 520 and communicating electronically with the monitoring system control unit 510 and monitoring server 560.

The predictive wellness engine 557 receives data from one or more sensors 520. In some examples, the predictive wellness engine 557 can be used to determine or indicate whether a user 108 is engaging in normal activity or whether an abnormal event has been detected that indicates the user 108 is at risk for a medical emergency or is experiencing an adverse wellness event based on data generated by sensors 520 (e.g., data from sensor 520 describing motion of mobile device 140, movement of the user 108, acceleration/velocity, orientation, and other parameters associated with the user 108 or their mobile device 140). The predictive wellness engine 557 can receive data from the one or more sensors 520 through any combination of wired and/or wireless data links. For example, the predictive wellness engine 557 can receive sensor data via a Bluetooth, Bluetooth LE, Z-wave, or Zigbee data link.

The predictive wellness engine 557 communicates electronically with the control unit 510. For example, the predictive wellness engine 557 can send data related to the sensors 520 to the control unit 510 and receive commands related to determining a state of mobile device 140 and wellness status of user 108 based on data from the sensors 520. In some examples, the predictive wellness engine 557 processes or generates sensor signal data, for signals emitted by the sensors 520, prior to sending it to the control unit 510. The sensor signal data can include information that indicates a user 108 has suddenly fallen, has been inactive and/or has not moved for a peculiar length of time, or has not charged their mobile device 140 in advance of an upcoming travel.

In some examples, the system 500 further includes one or more robotic devices 590. The robotic devices 590 may be any type of robots that are capable of moving and taking actions that assist in home monitoring. For example, the robotic devices 590 may include drones that are capable of moving throughout a home based on automated control technology and/or user input control provided by a user. In this example, the drones may be able to fly, roll, walk, or otherwise move about the home. The drones may include helicopter type devices (e.g., quad copters), rolling helicopter type devices (e.g., roller copter devices that can fly and also roll along the ground, walls, or ceiling) and land vehicle type devices (e.g., automated cars that drive around a home). In some cases, the robotic devices 590 may be devices that are intended for other purposes and merely associated with the system 500 for use in appropriate circumstances. For instance, a robotic vacuum cleaner device may be associated with the monitoring system 500 as one of the robotic devices 590 and may be controlled to take action responsive to monitoring system events.

In some examples, the robotic devices 590 automatically navigate within a home as well as outside a home. In these examples, the robotic devices 590 include sensors and control processors that guide movement of the robotic devices 590 within (or outside) the home. For instance, the robotic devices 590 may navigate within (or outside) the home using one or more cameras, one or more proximity sensors, one or more gyroscopes, one or more accelerometers, one or more magnetometers, a global positioning system (GPS) unit, an altimeter, one or more sonar or laser sensors, and/or any other types of sensors that aid in navigation about a space. The robotic devices 590 may include control processors that process output from the various sensors and control the robotic devices 590 to move along a path that reaches the desired destination and avoids obstacles. In this regard, the control processors detect walls or other obstacles in the home and guide movement of the robotic devices 590 in a manner that avoids the walls and other obstacles.

In addition, the robotic devices 590 may store data that describes attributes of the home. For instance, the robotic devices 590 may store a floorplan and/or a three-dimensional model of the home that enables the robotic devices 590 to navigate the home and the home's perimeter. During initial configuration, the robotic devices 590 may receive the data describing attributes of the home, determine a frame of reference to the data (e.g., a home or reference location in the home), and navigate the home based on the frame of reference and the data describing attributes of the home. Further, initial configuration of the robotic devices 590 also may include learning of one or more navigation patterns in which a user provides input to control the robotic devices 590 to perform a specific navigation action (e.g., fly to an upstairs bedroom and spin around while capturing video and then return to a home charging base). In this regard, the robotic devices 590 may learn and store the navigation patterns such that the robotic devices 590 may automatically repeat the specific navigation actions upon a later request.

In some examples, the robotic devices 590 may include data capture and recording devices. In these examples, the robotic devices 590 may include one or more cameras, one or more motion sensors, one or more microphones, one or more biometric data collection tools, one or more temperature sensors, one or more humidity sensors, one or more air flow sensors, and/or any other types of sensors that may be useful in capturing monitoring data related to the home and users in the home. The one or more biometric data collection tools may be configured to collect biometric samples of a person in the home with or without contact of the person. For instance, the biometric data collection tools may include a fingerprint scanner, a hair sample collection tool, a skin cell collection tool, and/or any other tool that allows the robotic devices 590 to take and store a biometric sample that can be used to identify the person (e.g., a biometric sample with DNA that can be used for DNA testing).

In some implementations, the robotic devices 590 may include output devices. In these implementations, the robotic devices 590 may include one or more displays, one or more speakers, and/or any type of output devices that allow the robotic devices 590 to communicate information to a nearby user.

The robotic devices 590 also may include a communication module that enables the robotic devices 590 to communicate with the control unit 510, each other, and/or other devices. The communication module may be a wireless communication module that allows the robotic devices 590 to communicate wirelessly. For instance, the communication module may be a Wi-Fi module that enables the robotic devices 590 to communicate over a local wireless network at the home. The communication module further may be a 900 MHz wireless communication module that enables the robotic devices 590 to communicate directly with the control unit 510. Other types of short-range wireless communication protocols, such as Bluetooth, Bluetooth LE, Z-wave, Zigbee, etc., may be used to allow the robotic devices 590 to communicate with other devices in the home. In some implementations, the robotic devices 590 may communicate with each other or with other devices of the system 500 through the network 505.

The robotic devices 590 further may include processor and storage capabilities. The robotic devices 590 may include any suitable processing devices that enable the robotic devices 590 to operate applications and perform the actions described throughout this disclosure. In addition, the robotic devices 590 may include solid state electronic storage that enables the robotic devices 590 to store applications, configuration data, collected sensor data, and/or any other type of information available to the robotic devices 590.

The robotic devices 590 are associated with one or more charging stations. The charging stations may be located at predefined home base or reference locations in the home. The robotic devices 590 may be configured to navigate to the charging stations after completion of tasks needed to be performed for the monitoring system 500. For instance, after completion of a monitoring operation or upon instruction by the control unit 510, the robotic devices 590 may be configured to automatically fly to and land on one of the charging stations. In this regard, the robotic devices 590 may automatically maintain a fully charged battery in a state in which the robotic devices 590 are ready for use by the monitoring system 500.

The charging stations may be contact based charging stations and/or wireless charging stations. For contact based charging stations, the robotic devices 590 may have readily accessible points of contact that the robotic devices 590 are capable of positioning and mating with a corresponding contact on the charging station. For instance, a helicopter type robotic device may have an electronic contact on a portion of its landing gear that rests on and mates with an electronic pad of a charging station when the helicopter type robotic device lands on the charging station. The electronic contact on the robotic device may include a cover that opens to expose the electronic contact when the robotic device is charging and closes to cover and insulate the electronic contact when the robotic device is in operation.

For wireless charging stations, the robotic devices 590 may charge through a wireless exchange of power. In these cases, the robotic devices 590 need only locate themselves closely enough to the wireless charging stations for the wireless exchange of power to occur. In this regard, the positioning needed to land at a predefined home base or reference location in the home may be less precise than with a contact based charging station. Based on the robotic devices 590 landing at a wireless charging station, the wireless charging station outputs a wireless signal that the robotic devices 590 receive and convert to a power signal that charges a battery maintained on the robotic devices 590.

In some implementations, each of the robotic devices 590 has a corresponding and assigned charging station such that the number of robotic devices 590 equals the number of charging stations. In these implementations, the robotic devices 590 always navigate to the specific charging station assigned to that robotic device. For instance, a first robotic device may always use a first charging station and a second robotic device may always use a second charging station.

In some examples, the robotic devices 590 may share charging stations. For instance, the robotic devices 590 may use one or more community charging stations that are capable of charging multiple robotic devices 590. The community charging station may be configured to charge multiple robotic devices 590 in parallel. The community charging station may be configured to charge multiple robotic devices 590 in serial such that the multiple robotic devices 590 take turns charging and, when fully charged, return to a predefined home base or reference location in the home that is not associated with a charger. The number of community charging stations may be less than the number of robotic devices 590.

Also, the charging stations may not be assigned to specific robotic devices 590 and may be capable of charging any of the robotic devices 590. In this regard, the robotic devices 590 may use any suitable, unoccupied charging station when not in use. For instance, when one of the robotic devices 590 has completed an operation or is in need of battery charge, the control unit 510 references a stored table of the occupancy status of each charging station and instructs the robotic device to navigate to the nearest charging station that is unoccupied.

The system 500 further includes one or more integrated security devices 580. The one or more integrated security devices may include any type of device used to provide alerts based on received sensor data. For instance, the one or more control units 510 may provide one or more alerts to the one or more integrated security input/output devices 580. Additionally, the one or more control units 510 may receive one or more sensor data from the sensors 520 and determine whether to provide an alert to the one or more integrated security input/output devices 580.

The sensors 520, the home automation controls 522, the camera 530, the thermostat 534, and the integrated security devices 580 may communicate with the controller 512 over communication links 524, 526, 528, 532, 538, 536, and 584. The communication links 524, 526, 528, 532, 538, and 584 may be a wired or wireless data pathway configured to transmit signals from the sensors 520, the home automation controls 522, the camera 530, the thermostat 534, and the integrated security devices 580 to the controller 512. The sensors 520, the home automation controls 522, the camera 530, the thermostat 534, and the integrated security devices 580 may continuously transmit sensed values to the controller 512, periodically transmit sensed values to the controller 512, or transmit sensed values to the controller 512 in response to a change in a sensed value.

The communication links 524, 526, 528, 532, 538, and 584 may include a local network. The sensors 520, the home automation controls 522, the camera 530, the thermostat 534, and the integrated security devices 580, and the controller 512 may exchange data and commands over the local network. The local network may include 802.11 “Wi-Fi” wireless Ethernet (e.g., using low-power Wi-Fi chipsets), Z-Wave, Zigbee, Bluetooth, “Homeplug” or other “Powerline” networks that operate over AC wiring, and a Category 5 (CAT5) or Category 6 (CAT6) wired Ethernet network. The local network may be a mesh network constructed based on the devices connected to the mesh network.

The monitoring server 560 is an electronic device configured to provide monitoring services by exchanging electronic communications with the control unit 510, the one or more user devices 540 and 550, and the central alarm station server 570 over the network 505. For example, the monitoring server 560 may be configured to monitor events (e.g., alarm events) generated by the control unit 510. In this example, the monitoring server 560 may exchange electronic communications with the network module 514 included in the control unit 510 to receive information regarding events (e.g., alerts) detected by the control unit 510. The monitoring server 560 also may receive information regarding events (e.g., alerts) from the one or more user devices 540 and 550.

In some examples, the monitoring server 560 may route alert data received from the network module 514 or the one or more user devices 540 and 550 to the central alarm station server 570. For example, the monitoring server 560 may transmit the alert data to the central alarm station server 570 over the network 505.

The monitoring server 560 may store sensor and image data received from the monitoring system and perform analysis of sensor and image data received from the monitoring system. Based on the analysis, the monitoring server 560 may communicate with and control aspects of the control unit 510 or the one or more user devices 540 and 550.

The monitoring server 560 may provide various monitoring services to the system 500. For example, the monitoring server 560 may analyze the sensor, image, and other data to determine an activity pattern of a resident of the home monitored by the system 500. In some implementations, the monitoring server 560 may analyze the data for alarm conditions or may determine and perform actions at the home by issuing commands to one or more of the controls 522, possibly through the control unit 510.

The central alarm station server 570 is an electronic device configured to provide alarm monitoring service by exchanging communications with the control unit 510, the one or more mobile devices 540 and 550, and the monitoring server 560 over the network 505. For example, the central alarm station server 570 may be configured to monitor alerting events generated by the control unit 510. In this example, the central alarm station server 570 may exchange communications with the network module 514 included in the control unit 510 to receive information regarding alerting events detected by the control unit 510. The central alarm station server 570 also may receive information regarding alerting events from the one or more mobile devices 540 and 550 and/or the monitoring server 560.

The central alarm station server 570 is connected to multiple terminals 572 and 574. The terminals 572 and 574 may be used by operators to process alerting events. For example, the central alarm station server 570 may route alerting data to the terminals 572 and 574 to enable an operator to process the alerting data. The terminals 572 and 574 may include general-purpose computers (e.g., desktop personal computers, workstations, or laptop computers) that are configured to receive alerting data from a server in the central alarm station server 570 and render a display of information based on the alerting data. For instance, the controller 512 may control the network module 514 to transmit, to the central alarm station server 570, alerting data indicating that a sensor 520 detected motion from a motion sensor via the sensors 520. The central alarm station server 570 may receive the alerting data and route the alerting data to the terminal 572 for processing by an operator associated with the terminal 572. The terminal 572 may render a display to the operator that includes information associated with the alerting event (e.g., the lock sensor data, the motion sensor data, the contact sensor data, etc.) and the operator may handle the alerting event based on the displayed information.

In some implementations, the terminals 572 and 574 may be mobile devices or devices designed for a specific function. Although FIG. 5 illustrates two terminals for brevity, actual implementations may include more (and, perhaps, many more) terminals.

The one or more authorized user devices 540 and 550 are devices that host and display user interfaces. For instance, the user device 540 is a mobile device that hosts or runs one or more native applications (e.g., the smart home application 542). The user device 540 may be a cellular phone or a non-cellular locally networked device with a display. The user device 540 may include a cell phone, a smart phone, a tablet PC, a personal digital assistant (“PDA”), or any other portable device configured to communicate over a network and display information. For example, implementations may also include Blackberry-type devices (e.g., as provided by Research in Motion), electronic organizers, iPhone-type devices (e.g., as provided by Apple), iPod devices (e.g., as provided by Apple) or other portable music players, other communication devices, and handheld or portable electronic devices for gaming, communications, and/or data organization. The user device 540 may perform functions unrelated to the monitoring system, such as placing personal telephone calls, playing music, playing video, displaying pictures, browsing the Internet, maintaining an electronic calendar, etc.

The user device 540 includes a smart home application 542. The smart home application 542 refers to a software/firmware program running on the corresponding mobile device that enables the user interface and features described throughout. The user device 540 may load or install the smart home application 542 based on data received over a network or data received from local media. The smart home application 542 runs on mobile devices platforms, such as iPhone, iPod touch, Blackberry, Google Android, Windows Mobile, etc. The smart home application 542 enables the user device 540 to receive and process image and sensor data from the monitoring system.

The user device 550 may be a general-purpose computer (e.g., a desktop personal computer, a workstation, or a laptop computer) that is configured to communicate with the monitoring server 560 and/or the control unit 510 over the network 505. The user device 550 may be configured to display a smart home user interface 552 that is generated by the user device 550 or generated by the monitoring server 560. For example, the user device 550 may be configured to display a user interface (e.g., a web page) provided by the monitoring server 560 that enables a user to perceive images captured by the camera 530 and/or reports related to the monitoring system. Although FIG. 5 illustrates two user devices for brevity, actual implementations may include more (and, perhaps, many more) or fewer user devices.

In some implementations, the one or more user devices 540 and 550 communicate with and receive monitoring system data from the control unit 510 using the communication link 538. For instance, the one or more user devices 540 and 550 may communicate with the control unit 510 using various local wireless protocols such as Wi-Fi, Bluetooth, Z-wave, Zigbee, HomePlug (Ethernet over power line), or wired protocols such as Ethernet and USB, to connect the one or more user devices 540 and 550 to local security and automation equipment. The one or more user devices 540 and 550 may connect locally to the monitoring system and its sensors and other devices. The local connection may improve the speed of status and control communications because communicating through the network 505 with a remote server (e.g., the monitoring server 560) may be significantly slower.

Although the one or more user devices 540 and 550 are shown as communicating with the control unit 510, the one or more user devices 540 and 550 may communicate directly with the sensors and other devices controlled by the control unit 510. In some implementations, the one or more user devices 540 and 550 replace the control unit 510 and perform the functions of the control unit 510 for local monitoring and long range/offsite communication.

In other implementations, the one or more user devices 540 and 550 receive monitoring system data captured by the control unit 510 through the network 505. The one or more user devices 540, 550 may receive the data from the control unit 510 through the network 505 or the monitoring server 560 may relay data received from the control unit 510 to the one or more user devices 540 and 550 through the network 505. In this regard, the monitoring server 560 may facilitate communication between the one or more user devices 540 and 550 and the monitoring system.

In some implementations, the one or more user devices 540 and 550 may be configured to switch whether the one or more user devices 540 and 550 communicate with the control unit 510 directly (e.g., through link 538) or through the monitoring server 560 (e.g., through network 505) based on a location of the one or more user devices 540 and 550. For instance, when the one or more user devices 540 and 550 are located close to the control unit 510 and in range to communicate directly with the control unit 510, the one or more user devices 540 and 550 use direct communication. When the one or more user devices 540 and 550 are located far from the control unit 510 and not in range to communicate directly with the control unit 510, the one or more user devices 540 and 550 use communication through the monitoring server 560.

Although the one or more user devices 540 and 550 are shown as being connected to the network 505, in some implementations, the one or more user devices 540 and 550 are not connected to the network 505. In these implementations, the one or more user devices 540 and 550 communicate directly with one or more of the monitoring system components and no network (e.g., Internet) connection or reliance on remote servers is needed.

In some implementations, the one or more user devices 540 and 550 are used in conjunction with only local sensors and/or local devices in a house. In these implementations, the system 500 includes the one or more user devices 540 and 550, the sensors 520, the home automation controls 522, the camera 530, the robotic devices 590, and the predictive wellness engine 557. The one or more user devices 540 and 550 receive data directly from the sensors 520, the home automation controls 522, the camera 530, the robotic devices 590, and the predictive wellness engine 557 and sends data directly to the sensors 520, the home automation controls 522, the camera 530, the robotic devices 590, and the predictive wellness engine 557. The one or more user devices 540, 550 provide the appropriate interfaces/processing to provide visual surveillance and reporting.

In other implementations, the system 500 further includes network 505 and the sensors 520, the home automation controls 522, the camera 530, the thermostat 534, the robotic devices 590, and the predictive wellness engine 557 are configured to communicate sensor and image data to the one or more user devices 540 and 550 over network 505 (e.g., the Internet, cellular network, etc.). In yet another implementation, the sensors 520, the home automation controls 522, the camera 530, the thermostat 534, the robotic devices 590, and the predictive wellness engine 557 (or a component, such as a bridge/router) are intelligent enough to change the communication pathway from a direct local pathway when the one or more user devices 540 and 550 are in close physical proximity to the sensors 520, the home automation controls 522, the camera 530, the thermostat 534, the robotic devices 590, and the predictive wellness engine 557 to a pathway over network 505 when the one or more user devices 540 and 550 are farther from the sensors 520, the home automation controls 522, the camera 530, the thermostat 534, the robotic devices 590, and the predictive wellness engine.

In some examples, the system leverages GPS information from the one or more user devices 540 and 550 to determine whether the one or more user devices 540 and 550 are close enough to the sensors 520, the home automation controls 522, the camera 530, the thermostat 534, the robotic devices 590, and the predictive wellness engine 557 to use the direct local pathway or whether the one or more user devices 540 and 550 are far enough from the sensors 520, the home automation controls 522, the camera 530, the thermostat 534, the robotic devices 590, and the predictive wellness engine 557 that the pathway over network 505 is required.

In other examples, the system leverages status communications (e.g., pinging) between the one or more user devices 540 and 550 and the sensors 520, the home automation controls 522, the camera 530, the thermostat 534, the robotic devices 590, and the predictive wellness engine 557 to determine whether communication using the direct local pathway is possible. If communication using the direct local pathway is possible, the one or more user devices 540 and 550 communicate with the sensors 520, the home automation controls 522, the camera 530, the thermostat 534, the robotic devices 590, and the predictive wellness engine 557 using the direct local pathway. If communication using the direct local pathway is not possible, the one or more user devices 540 and 550 communicate with the sensors 520, the home automation controls 522, the camera 530, the thermostat 534, the robotic devices 590, and the predictive wellness engine 557 using the pathway over network 505.

In some implementations, the system 500 provides end users with access to images captured by the camera 530 to aid in decision making. The system 500 may transmit the images captured by the camera 530 over a wireless WAN network to the user devices 540 and 550. Because transmission over a wireless WAN network may be relatively expensive, the system 500 can use several techniques to reduce costs while providing access to significant levels of useful visual information (e.g., compressing data, down-sampling data, sending data only over inexpensive LAN connections, or other techniques).

In some implementations, a state of the monitoring system and other events sensed by the monitoring system may be used to enable/disable video/image recording devices (e.g., the camera 530). In these implementations, the camera 530 may be set to capture images on a periodic basis when the alarm system is armed in an “away” state, but set not to capture images when the alarm system is armed in a “home” state or disarmed. In addition, the camera 530 may be triggered to begin capturing images when the alarm system detects an event, such as an alarm event, a door-opening event for a door that leads to an area within a field of view of the camera 530, or motion in the area within the field of view of the camera 530. In other implementations, the camera 530 may capture images continuously, but the captured images may be stored or transmitted over a network when needed.

The described systems, methods, and techniques may be implemented in digital electronic circuitry, computer hardware, firmware, software, or in combinations of these elements. Apparatus implementing these techniques may include appropriate input and output devices, a computer processor, and a computer program product tangibly embodied in a machine-readable storage device for execution by a programmable processor. A process implementing these techniques may be performed by a programmable processor executing a program of instructions to perform desired functions by operating on input data and generating appropriate output. The techniques may be implemented in one or more computer programs that are executable on a programmable system including at least one programmable processor coupled to receive data and instructions from, and to transmit data and instructions to, a data storage system, at least one input device, and at least one output device.

Each computer program may be implemented in a high-level procedural or object-oriented programming language, or in assembly or machine language if desired; and in any case, the language may be a compiled or interpreted language. Suitable processors include, by way of example, both general and special purpose microprocessors. Generally, a processor will receive instructions and data from a read-only memory and/or a random access memory.

Storage devices suitable for tangibly embodying computer program instructions and data include all forms of non-volatile memory, including by way of example semiconductor memory devices, such as Erasable Programmable Read-Only Memory (EPROM), Electrically Erasable Programmable Read-Only Memory (EEPROM), and flash memory devices; magnetic disks such as internal hard disks and removable disks; magneto-optical disks; and Compact Disc Read-Only Memory (CD-ROM). Any of the foregoing may be supplemented by, or incorporated in, specially designed ASICs (application-specific integrated circuits).

It will be understood that various modifications may be made. For example, other useful implementations could be achieved if steps of the disclosed techniques were performed in a different order and/or if components in the disclosed systems were combined in a different manner and/or replaced or supplemented by other components. Accordingly, other implementations are within the scope of the disclosure.

1-20. (canceled)

21. A computer-implemented method, comprising:
maintaining sensor data generated by one or more sensors that indicates one or more characteristics of a person;
maintaining a predictive model for the person that is configured to process sensor data that indicates characteristics of the person to compute one or more patterns of the person;
updating an activity profile of the person that indicates the one or more patterns of the person by processing the sensor data using the predictive model;
determining one or more actions to perform for the person by processing the activity profile of the person; and
causing performance of the one or more actions.

22. The method of claim 21, wherein determining the one or more actions to perform for the person by processing the activity profile of the person comprises:
detecting, using the activity profile of the person, an abnormal event for the person; and
determining the one or more actions using data that indicates the abnormal event for the person.

23. The method of claim 22, wherein:
the activity profile of the person comprises one or more threshold values for abnormal events involving the person; and
detecting the abnormal event for the person comprises detecting an occurrence of an event that satisfies at least one of the one or more threshold values.

24. The method of claim 22, wherein:
one of the one or more actions comprises generating a notification for the abnormal event; and
causing performance of the one or more actions comprises generating a notification for the abnormal event.

25. The method of claim 22, wherein causing performance of the one or more actions comprises, in response to detecting the abnormal event, initiating a voice connection between a property where the person is located and a central monitoring station that monitors the property.

26. The method of claim 21, wherein:
one of the one or more actions comprises generating, using the activity profile of the person, instructions for presentation of a graphical interface that indicates a current health condition of the person; and
causing performance of the one or more actions comprises generating, using the activity profile of the person, instructions for presentation of a graphical interface that indicates a current health condition of the person.

27. The method of claim 21, wherein:
at least some of the one or more sensors are integrated into a mobile device of the person, the method comprising:
receiving, from the mobile device of the person, at least some of the sensor data that was generated by the at least some of the one or more sensors integrated into the mobile device.

28. The method of claim 21, wherein:
the activity profile comprises parameter values that are indicative of normal activity of the person;
at least one of the parameter values of the activity profile indicates a rate of physical activity of the person; and
determining the one or more actions to perform for the person by processing the activity profile of the person comprises determining the one or more actions using the at least one of the parameter values that indicates a rate of physical activity of the person.

29. A system comprising a processing device and a non-transitory machine-readable storage device storing instructions that are executable by the processing device to cause performance of operations comprising:
maintaining sensor data generated by one or more sensors that indicates one or more characteristics of a person;
maintaining a predictive model for the person that is configured to process sensor data that indicates characteristics of the person to compute one or more patterns of the person;
updating an activity profile of the person that indicates the one or more patterns of the person by processing the sensor data using the predictive model;
determining one or more actions to perform for the person by processing the activity profile of the person; and
causing performance of the one or more actions.

30. The system of claim 29, wherein determining the one or more actions to perform for the person by processing the activity profile of the person comprises:
detecting, using the activity profile of the person, an abnormal event for the person; and
determining the one or more actions using data that indicates the abnormal event for the person.

31. The system of claim 30, wherein:
the activity profile of the person comprises one or more threshold values for abnormal events involving the person; and
detecting the abnormal event for the person comprises detecting an occurrence of an event that satisfies at least one of the one or more threshold values.

32. The system of claim 30, wherein:
one of the one or more actions comprises generating a notification for the abnormal event; and
causing performance of the one or more actions comprises generating a notification for the abnormal event.

33. The system of claim 30, wherein causing performance of the one or more actions comprises, in response to detecting the abnormal event, initiating a voice connection between a property where the person is located and a central monitoring station that monitors the property.

34. The system of claim 29, wherein:
one of the one or more actions comprises generating, using the activity profile of the person, instructions for presentation of a graphical interface that indicates a current health condition of the person; and
causing performance of the one or more actions comprises generating, using the activity profile of the person, instructions for presentation of a graphical interface that indicates a current health condition of the person.

35. The system of claim 29, wherein:
at least some of the one or more sensors are integrated into a mobile device of the person, the operations comprising:
receiving, from the mobile device of the person, at least some of the sensor data that was generated by the at least some of the one or more sensors integrated into the mobile device.

36. The system of claim 29, wherein:
the activity profile comprises parameter values that are indicative of normal activity of the person;
at least one of the parameter values of the activity profile indicates a rate of physical activity of the person; and
determining the one or more actions to perform for the person by processing the activity profile of the person comprises determining the one or more actions using the at least one of the parameter values that indicates a rate of physical activity of the person.

37. A non-transitory machine-readable storage device storing instructions that are executable by a processing device to cause performance of operations comprising:
maintaining sensor data generated by one or more sensors that indicates one or more characteristics of a person;
maintaining a predictive model for the person that is configured to process sensor data that indicates characteristics of the person to compute one or more patterns of the person;
updating an activity profile of the person that indicates the one or more patterns of the person by processing the sensor data using the predictive model;
determining one or more actions to perform for the person by processing the activity profile of the person; and
causing performance of the one or more actions.

38. The machine-readable storage device of claim 37, wherein determining the one or more actions to perform for the person by processing the activity profile of the person comprises:
detecting, using the activity profile of the person, an abnormal event for the person; and
determining the one or more actions using data that indicates the abnormal event for the person.

39. The machine-readable storage device of claim 38, wherein:
the activity profile of the person comprises one or more threshold values for abnormal events involving the person; and
detecting the abnormal event for the person comprises detecting an occurrence of an event that satisfies at least one of the one or more threshold values.

40. The machine-readable storage device of claim 38, wherein:
one of the one or more actions comprises generating a notification for the abnormal event; and
causing performance of the one or more actions comprises generating a notification for the abnormal event.
